(19) United States
(12) Patent Application Publication
(10) Pub. No.: US 2010/0198262 A1
(43) Pub. Date: Aug. 5, 2010

(54) AXIAL OFFSET BONE FASTENER SYSTEM

(76) Inventor: Laurence M. McKinley, Escondido, CA (US)

Correspondence Address: KAUTH , POMEROY , PECK & BAILEY ,LLP, 2875 MICHELLE DRIVE, SUITE 110, IRVINE, CA, 92606, US

(21) Appl. No.: 12/362,739

(22) Filed: Jan. 30, 2009

(51) Int. Cl. A61B 17/70; A61B 17/86

(52) U.S. Cl. 606/265; 606/301

(57) ABSTRACT

A bone fastener assembly and a method of stabilizing a bone using such an assembly are presented. The bone assembly includes a bone fastener and a connecting member for holding a stabilizing member, such as a rod or plate, mounted together via an armature that allows the connecting member to be offset from the longitudinal axis of the bone fastener and optionally also permits the bone fastener to pivot relative to a longitudinal axis of the assembly and the connecting member swivel about the longitudinal axis of the assembly. By allowing the connecting member to be offset and independently swivel about the longitudinal axis of the fastener, the bone assembly of the current invention allows for an unprecedented degree of freedom in positioning an interconnecting rod to that assembly.

### CROSS-REFERENCE TO RELATED APPLICATIONS

The present application claims priority to U.S. Provisional Patent No. 60/024,629, filed Jan. 30, 2008, the disclosure of which is incorporated herein by reference.

### FIELD OF THE INVENTION

The current invention is directed to a bone fastener system; and more specifically to a bone fastener system having a stabilizing member connector that is offset from the axis of the fastener.

### BACKGROUND OF THE INVENTION

A multitude of orthopedic and neurological procedures use fasteners of one kind or another to connect one bone, or bone fragment, to another. Among these the most common use is in the connection of one vertebra of the human spine to another vertebra. In particular, the vertebrae of the human spine are positioned in a delicate arrangement that evenly distributes the force between the adjacent vertebrae and allows the spine to flex and twist with remarkable freedom. However, this delicate arrangement can be easily disrupted by a number of external stresses such as age, trauma, disease or general malformation.

Most conventional fastener systems operate through a plurality of bone fasteners interconnected by stabilizing rods. Although the specific design of these fasteners and rods have taken a number of different forms, in the vast majority of these systems the rod is held by a rod engagement seat that is positioned along the axis of the fastener. While this arrangement provides excellent support in helping fuse adjacent vertebrae in most cases, particularly in relatively simple deformities where the spine is still in a conventional conformation, these fasteners are not well engineered in complex cases where spinal fusion is combined with neurological decompression.

Specifically, it is often the case that there are several goals in performing an operation on an individual. For example, sometimes it is necessary to both decompress the nerves around a particular vertebral body and perform a fusion on a badly deformed spine. In this case, it is important that the rods interconnecting the vertebral bodies not encroach over the top of the spinal decompression because with the twists and bends found in some badly deformed spines it is possible that the metal of the rods can come close enough to impinge on the nearby nerves causing chronic pain and in some cases failure to fuse. However, in conventional systems, because the rod's position in relation to the vertebral fastener cannot be adjusted, it is necessary to radically bend and twist the rod to avoid this encroachment. Unfortunately, it is known that such radical bending and twisting can damage the crystalline structure of the metal in the rods, which in turn can lead to pre-stressing of the material and premature failure of the rods.

Accordingly, a need exists for an improved fastener system that allows for more flexibility in the positioning of the rod in relation to the vertebral fastener itself.

### SUMMARY OF THE INVENTION

The current invention is directed to a bone fastener system incorporating an axially offset stabilizing member assembly.

In one embodiment of the invention the bone fastener is screw shaft having a head that interconnects with connector seat.

In another embodiment of the invention the connector seat is designed to secure a rod assembly therein.

In still another embodiment of the invention the connector seat is hingedly interconnected with the fastener through an armature such that the connector seat may be adjusted after fixation on either side of the fastener axis.

In yet another embodiment of the invention the length of the armature interconnecting the connector seat to the fastener may be adjusted radially such that the distance of the connector seat relative to the fastener may be changed.

In still yet another embodiment of the invention the connector seat is rotatably connected to the fastener such that the angle of the rod relative to the fastener may be adjusted.

### BRIEF DESCRIPTION OF THE FIGURES

Various examples of the present invention will be discussed with reference to the appended drawings. These drawings depict only illustrative examples of the invention and are not to be considered limiting of its scope.

FIG. 1a is a perspective view of one embodiment of a bone fastener assembly according to the present invention;

FIG. 1b is a side view of the bone fastener shown in FIG. 1a;

FIG. 1c is a top view of the bone fastener shown in FIG. 1a;

FIG. 1d is an exploded view showing the interconnection between the rod connector and the fastener body of the bone fastener shown in FIG. 1a;

FIG. 2 shows a side view of a second embodiment of a bone fastener assembly according to the present invention;

FIG. 3 shows a side view of a third embodiment of a bone fastener assembly according to the present invention;

FIG. 4 shows a side view of a fourth embodiment of a bone fastener assembly according to the present invention; and

FIG. 5 shows a top view of a variable offset embodiment of a bone fastener assembly according to the present invention.

### DETAILED DESCRIPTION OF THE INVENTION

Embodiments of a bone fastener assembly according to the present invention include a bone fastener and a connecting member for holding a stabilizing member, such as a rod or plate, mounted together via an armature that allows the connecting member to be offset from the longitudinal axis of the bone fastener and optionally also permits the bone fastener to pivot relative to a longitudinal axis of the assembly and the connecting member swivel about the longitudinal axis of the assembly. By allowing the connecting member to be offset and independently swivel about the longitudinal axis of the assembly, the bone fastener assembly provides an unprecedented degree of freedom in positioning an interconnecting rod to that assembly.

### Definitions

Bone Fastener: For the purposes of this invention, a bone fastener is defined as any device suitable for engaging a bone, including, a screw, pin, nail, bolt, staple, hook, etc.

Stabilizing Member: For the purposes of this invention, a stabilizing member is defined as any device suitable for interconnecting and stabilizing bone, including, plates, rods, etc.

Connecting Member: For the purposes of this invention, a connecting member may include any suitable member for assembling a construct for stabilizing bones of a patient such as a plate engaging stud, rod holder, etc.

### Description

The bone fastener assembly of the current invention is comprised of three fundamental parts: a bone fastener for anchoring the assembly to a bone, a rotatable connecting member for holding a stabilizing member such as a plate rod, etc., and an offset linkage interconnecting the bone fastener to the connecting member. The following description details exemplary embodiments of these bone fastener assembly of the current invention.

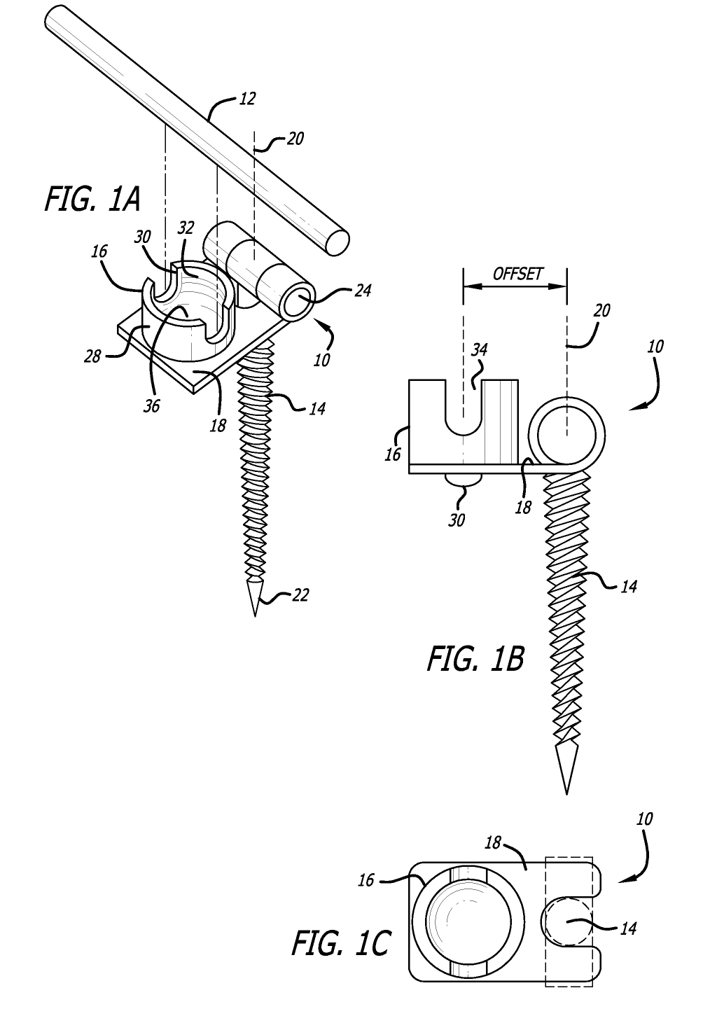

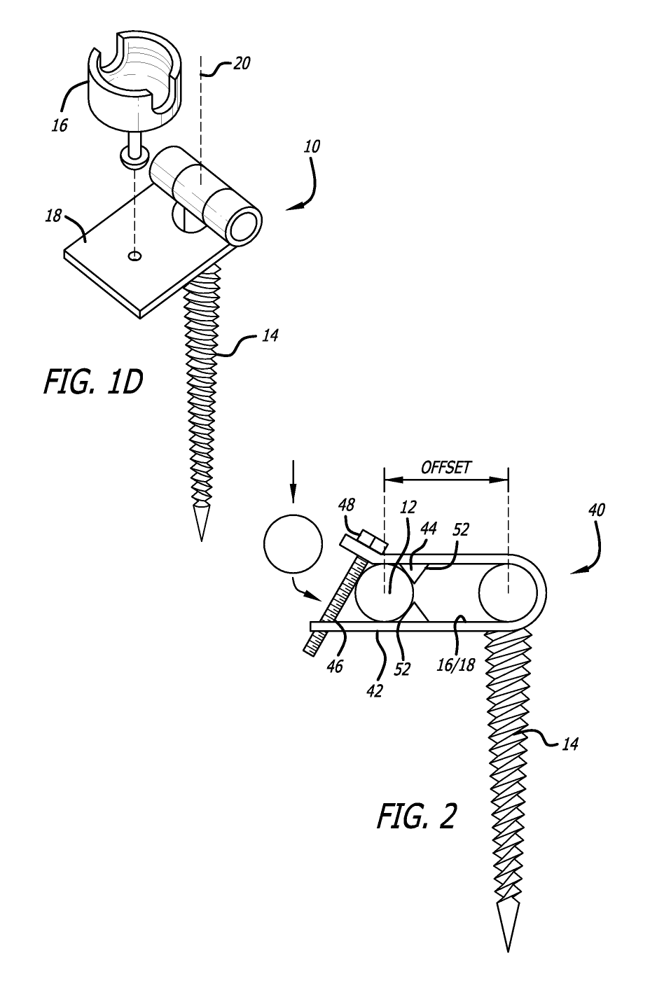

FIGS. 1a to 1d illustrate a bone fastener assembly in the form of a pedicle screw assembly 10 coupled to a spinal rod 12. As shown, each pedicle screw assembly 10 includes a bone fastener in the form of a screw 14 having a “T” shaped head, a connecting member in the form of a rod holder 16, and a offset linkage in the form of a hinged armature 18 (FIG. 1a) pivotally linking the screw 14 and rod holder 16. The assembly includes a longitudinal axis 20 through the rod holder 16 and screw 14.

As best seen in FIG. 1a, the offset linkage 18 is, in one embodiment, able to pivot azimuthally in a vertical plane about the bone fastener 10 such that the side of the bone fastener on which the offset of the connecting member 16 is disposed, and the angle between the connecting member 16 and the longitudinal axis 20 of the screw 14 may be altered. In addition to the hinged offset linkage 18, which allows the angle of the connecting member 16 to be varied azimuthally in relation to the longitudinal axis 20 of the screw 14, as best seen in FIGS. 1b to 1d, in one embodiment the connecting member 16 may also engage the offset linkage 18 through a swivel connection, which allows for the connecting member to be rotated about the longitudinal axis 20 in a plane horizontal to the longitudinal axis 20 of the bone screw 10. This combination of offset, vertical pivoting and horizontal swiveling allows the connecting member rod holder 16 to be moved to any desired position independent of the position of the screw 14 itself. Various mechanisms for achieving this range of motion are detailed below.

First, with regard to the exemplary embodiment shown in FIGS. 1a to 1d, in this first embodiment the distance of the offset is fixed by the length of the offset linkage 18. In addition, in this first embodiment the connecting member 16 is rotatably connected to the offset linkage to provide yet another degree of freedom in positioning the rod in relation to the overall assembly. However, it should be understood that these “degrees of freedom” are not necessary to the invention. For example, the connecting member 16 could be fixedly attached to the offset linkage 18 and the offset linkage 18 itself could be fixedly attached to the bone fastener 14.

As discussed above, although any suitable bone fastener may be used, in one embodiment the screw 14 includes an elongated shank 21 having a tip 22 at a distal end, a head 24 at a proximal end, and a longitudinal axis 20 extending therebetween. A thread 26 spirals around the shank such that the screw 14 may be threaded into a bone. As shown in FIG. 1, in a preferred embodiment the head 24 is a “T”-shape formed by the arrangement of a cylinder disposed transverse to the longitudinal axis 20 of the screw 14. However, it should be understood that any head shape may be used that allows the attachment of an offset linkage 18 may be use with the current invention.

Likewise, although FIG. 1 shows a preferred embodiment of a connecting member in accordance with the current invention, it should be understood that any connecting member that allows a rod, stud, etc. to be secured to the bone fastener may be used. In the embodiment shown in FIG. 1, the connecting member 16 includes a generally cylindrical body 28 having a transverse passageway 30 extends through the body for receiving a rod 12. Preferably, the transverse passageway 30 is open in a direction transverse to the axis of the rod 12 to allow the rod to be placed into the connecting member 16 with a single transverse motion. Alternatively, the transverse passageway 30 may be closed such that the rod 12 must be inserted through the connecting member 16 along the axis of the transverse passageway.

To secure the rod within the passageway 30 any suitable securing means may be used. As shown in FIG. 1, in one embodiment a screw thread 32 spirals from an upper opening 34 distally into the cylindrical body 28. The connecting member 16 has a lower contacting surface 36 for engaging the stabilizing means, in this case the rod. The rod holder contacting surface 36 is preferably convex and spherical to match the shape of the rod. A set screw (not shown) engages the thread 32 in the upper opening of the cylindrical body 28 of the connecting member 16.

Finally, interconnecting the connecting member 16 and the bone fastener 14 is an offset linkage 18. As shown in FIG. 1, the offset linkage generally comprises an elongated armature with an attachment point for the connecting means 16 at one end and means of connecting the armature to the head of the screw on the other. As shown, in the embodiment in FIG. 1 the means of connecting the armature to the head of the screw is a pivotable hinge that allows the offset linkage 18 and in turn the connecting means 16 to pivot azimuthally from one side of the longitudinal axis of the bone fastener 14 to the other. As shown in FIG. 1d, in such an embodiment the connecting means 16 may be removably engaged to the offset linkage 18 such that it may be inserted on the top surface of the linkage regardless of which side of the screw the linkage is positioned on.

FIGS. 2 to 4 illustrate a number of alternative embodiments of the offset bone fastener assembly of the current invention that is generally configured like, and operates like the screw seat 10 of FIG. 1. Specifically, fastener assembly 40 differs from that of FIG. 1, in that the connecting member 16 is made as an integral part of the offset linkage 18. Specifically, in the embodiment shown in each of these figures the offset linkage 18 is in the form of a “U” shaped stirrup that is connected to the head of the screw at the closed end and has a rod retaining means 42 at the open end. The closed end of the “U” shaped member may either be rotatably connected to the head of the screw or may alternatively be fixedly attached to the head of the screw.

Turning to the rod attachment end 42 of the offset linkage, as shown in FIGS. 2 to 4 a number of possible alternatives may be used; however, each of these attachment ends have some common features. First, the rod attachment end 42 of the offset linkage generally comprises two arms, upper 44 and lower 46, arranged parallel to each other and transverse to the axial direction of the rod 12. To engage the rod the upper and lower arms are urged together through a suitable fixation means, such as for example a screw 48 that engages threaded openings in both arms such that when the screw is tightened the arms are brought together. It should be understood that though tightening screws 48 are shown for each of the embodiments shown in FIGS. 2 to 4, it should be understood that any suitable means of urging the upper and lower arms together to engage the rod 12 may be used.

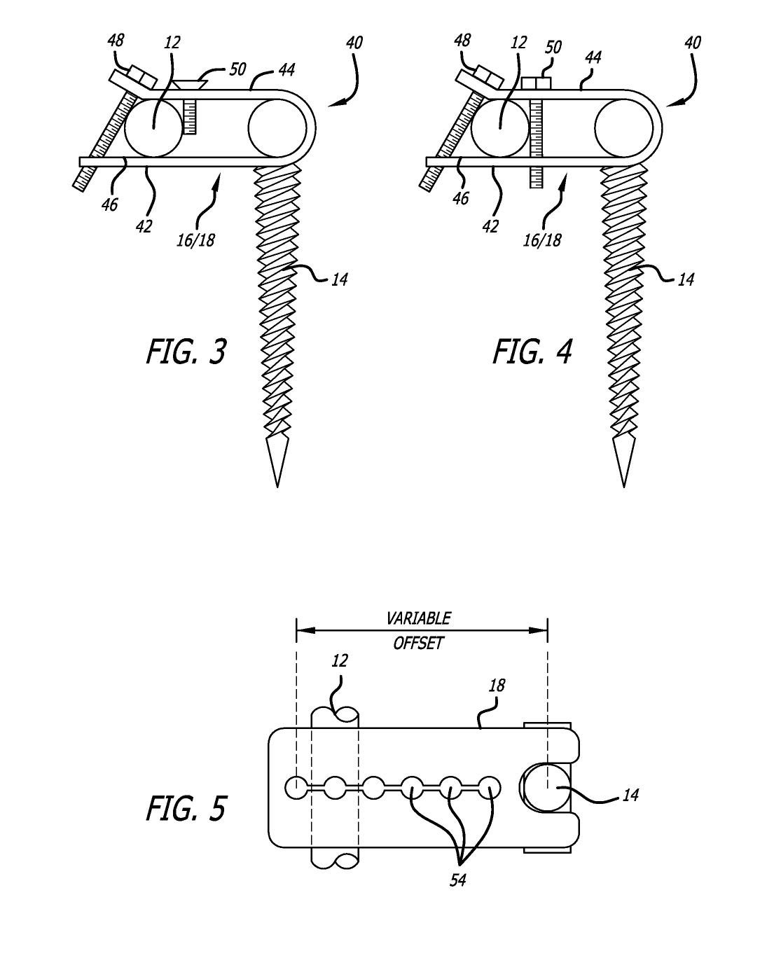

Although the engaging or tightening screw 48 engages the rod 12 between the arms 44 and 46 to prevent the rod from slipping from between the armatures, because the armatures are aligned parallel, absent some inner retaining means it would still be possible for the rod to move along the arms 44 and 46 inward toward the screw 14. Accordingly, some inner retaining means is required to ensure stable fixation of the rod 12 within the rod attachment end 42 of the offset Linkage 18. A number of exemplary alternatives are shown in FIGS. 2 to 4, these will be discussed further in the following section. First, in FIG. 2 an exemplary embodiment is shown in which the inner retaining means 50 comprises divots 52 formed into the upper 44 and Lower 46 arms. It should be understood that while the embodiment shown in FIG. 2 shows divots formed in both the upper and lower arms, that a single Larger divot could be formed on only one of the arms to serve the same purpose. FIG. 3 shows a second exemplary embodiment where the inner retaining means 50 comprises a simple set screw. Again, although in the embodiment shown the set screw is positioned in the upper arm 44, it should be understood that the set screw may be positioned in either of the arms 44 and 46. Finally, although the set screw shown in FIG. 3 only engages one of the arms, such a screw could be made to engage both of the armatures as shown in FIG. 4.

Finally, although all of the embodiments of the offset bone fastener system shown thus far have had a fixed offset distance, that is the offset distance is predetermined by the length of the offset linkage, it should be understood that the current invention also contemplates variable offset systems. First, in the embodiments shown in FIGS. 2 to 4, a plurality of different interchangeable Linkage Lengths might be supplied with the bone fastener system. In such an embodiment the practitioner would determine the desired length, attach the correct offset linkage 18 to the screw 14 when placing the fastener into the patient. However, obviously such a system is still limited in that it would be difficult to interchange such linkages during a surgical procedure. FIG. 5 illustrates an alternative embodiment of the fastener system of the current invention having an adjustable offset linkage 18. As shown, in this embodiment the offset linkage 18 is provided with a plurality of threaded openings 54 arranged along the top of the arm of the offset linkage 18. Such an arrangement allows for the offset of the rod 12 to be adjusted inward or outward transverse to the longitudinal axis of the screw by positioning outer and inner set screws at desired positions along the linkage 18. Using such a system it is possible to provide an adjustable offset

Although the above has only focused on the construction of the bone fastener assembly and system of the current invention, it should be understood that the invention is also directed to a method of stabilizing a bone using the system described herein. For example using the embodiment shown in FIG. 1, during surgery, the screw 14 is driven into a bone, e.g. a pedicle, at a desired angle. The offset linkage 18 is pivoted and positioned relative to the screw 14 in a desired position, and then the rod holder 16 is swiveled relative to the screw 14 to a desired orientation to align the transverse passageway of the rod holder 16 with a desired rod orientation. The rod 12 is then placed into the rod holder 16 and secured by a screw into the threaded cylindrical body 28 to press the rod 12 and rod holder 16 together to lock the construct in the desired position. Referring to FIG. 5, in one embodiment, once the screw 14 and linkage offset 18 are positioned the offset of the rod holder 16 is adjusted and the rod 12 is inserted into the rod holder 16 is locked into the desired position.

Regardless of the specific embodiment, the screw 14, the connecting member/rod holder 16 and offset linkage 18 may be assembled intraoperatively by the surgical team or they may be preassembled. Preferably, the components are preassembled and locked in place to prevent disassembly in order to simplify their use in surgery.

While the specific embodiments used to illustrate the invention show the bone fastener assembly in the form of a pedicle screw useful to attach a rod to a vertebrae, the bone fastener assembly may connect to a bone in any of the variety of ways known in the art and may be utilized in any of the variety of constructs known in the art to stabilize bones at any location within the body.

Although examples of a bone fastener assembly and its use have been described and illustrated in detail, it is to be understood that the same is intended by way of illustration and example only and is not to be taken by way of limitation. The invention has been illustrated in the form of a pedicle screw assembly for use in assembling stabilizing constructs to connect vertebrae of the human spine. However, the bone fastener assembly may be configured with other kinds of fasteners and connecting members to assemble other kinds of constructs to stabilize bones and bone fragments at any location in the body. Accordingly, variations in and modifications to the bone fastener assembly and its use will be apparent to those of ordinary skill in the art. The various illustrative embodiments illustrate alternative configurations of various component parts such as screw seats, screws, pivot mechanisms, swivel mechanisms, and inserts among others. In most cases, and as will be readily understood by one skilled in the art, the alternative configuration of a component part in one embodiment may be substituted for a similar component part in another embodiment. Likewise, the various mechanisms illustrated for attaching the screw seat to the rod holder may be interchanged. Furthermore, throughout the exemplary embodiments, where component part mating relationships are illustrated, the gender of the component parts may be reversed as is known in the art within the scope of the invention. The following claims are intended to cover all such modifications and equivalents

What is claimed is:

1. An axially offset bone fastener system for connecting a bone to a spinal rod comprising:
a bone fastener having an elongated shank including a bone engaging portion at a first end, a head at an opposite, second end, and a longitudinal screw axis extending therebetween, the head being elongated in a direction transverse to the longitudinal screw axis such that the bone fastener is generally “T”-shaped;
a connecting member, having a longitudinal connecting member axis, and a rod receiving channel extending at least partially through the connecting member transverse to the longitudinal axis for receiving a spinal rod; and
an offset linkage interconnecting said connecting member and the head of the bone fastener, wherein the connecting member is disposed on the offset linkage such that the longitudinal connecting member axis is laterally offset from the longitudinal screw axis by a predefined distance.

2. The offset bone fastener system of claim 1, wherein the offset linkage is pivotally interconnected with said “T”-shaped head such that the connecting member azimuthally pivots about the head of said bone fastener.

3. The offset bone fastener system of claim 1, wherein the connecting member is rotatably mounted to the offset linkage such that the connecting member rotates about the longitudinal connecting member axis.

4. The offset bone fastener system of claim 1, wherein the offset linkage is pivotally interconnected with said “T”-shaped head such that the connecting member azimuthally pivots about the head of said bone fastener; and
wherein the connecting member is rotatably mounted to the offset Linkage such that the connecting member rotates about the longitudinal connecting member axis

5. The offset bone fastener system of claim 1, wherein the connecting member comprises a cylindrical body having a lower surface rotatably connected to the offset Linkage and a threading disposed on an upper surface, the connecting member defining a channel formed in the cylindrical body such that an elongated rod may be received therein, the connecting member further comprising a cooperatively threaded mating nut having a plurality of engaging teeth disposed on the Lower surface thereof and designed to frictionally Lock the rod within the channel of the connecting member.

6. The offset bone fastener system of claim 1, wherein the rod receiving channel extends fully through the connecting member.

7. The offset bone fastener system of claim 1, wherein the rod receiving channel defines a longitudinal channel axis transverse to the longitudinal connecting axis, and where the channel is at least partially open along said longitudinal channel axis.

8. The offset bone fastener system of claim 1, wherein the rod receiving channel defines a longitudinal channel axis transverse to the longitudinal connecting axis, and where the channel is fully enclosed along said longitudinal channel axis.

9. The offset bone fastener system of claim 1, wherein the assembly is made of stainless steel.

10. The offset bone fastener system of claim 1, wherein the head of the bone fastener further comprises a recess disposed in the head thereof and designed to receive a screw driver from the group consisting of: flat-headed, phillips head, allen wrench, and square headed.

11. The offset bone fastener system of claim 1, wherein the screw further comprises a slotted groove disposed longitudinally along the tip and designed to allow the screw to be self-tapped.

12. The offset bone fastener system of claim 1, wherein the connecting member and the offset linkage are a single integrated piece.

13. The offset bone fastener system of claim 11, wherein the single integrated piece is formed of two parallel and interconnected armatures, having a first closed end proximate to and interconnected with said bone fastener, and a second open end opposite the first end such that the single integrated piece is generally “U”-shaped.

14. The offset bone fastener system of claim 13, wherein the rod retaining channel is defined by the opening between the parallel armatures.

15. The offset bone fastener system of claim 13, wherein each of the armatures have at least one cooperative threaded opening, such that at least one threaded fastener may be engaged therebetween.

16. The offset bone fastener system of claim 13, wherein at least one of said armatures has a threaded opening disposed therein, such that at least one lateral side of the rod receiving channel may be defined by at least one removable fastener.

17. The offset bone fastener system of claim 16, wherein the at least one removable fastener is a screw.

18. The offset bone fastener system of claim 13, wherein at least one of said armatures has at least two threaded openings disposed therein, such that both lateral sides of the rod receiving channel may each be defined by at least one removable fastener.

19. The offset bone fastener system of claim 18, wherein the removable fasteners are a plurality of screw.

20. The offset bone fastener system of claim 19, wherein the at least one armatures comprises a plurality of threaded openings spaced across the length thereof such that the position of the rod receiving channel relative to the longitudinal screw axis may be varied.

21. The offset bone fastener system of claim 1, wherein the lateral offset distance between the longitudinal screw axis and longitudinal connecting member axis is fixed.

22. The offset bone fastener system of claim 1, wherein the lateral offset distance between the longitudinal screw axis and longitudinal connecting member axis is variable.

23. The offset bone fastener system of claim 22, wherein the lateral offset distance is defined by the relative position of at least two removable fasteners disposed in said offset linkage.

24. The offset bone fastener system of claim 23, wherein the at least two removable fasteners are screws.

25. The offset bone fastener system of claim 1, wherein the system comprises a plurality of interchangeably removable offset linkages each having a different predefined fixed offset distance.

26. A vertebral alignment/fixation method comprising utilizing a plurality of the offset bone fastener systems as described in claim 1 to align at Least one vertebral body.
